SEQUENCE LISTING (6 sequences)

SEQ ID NO: 1 - DNA, 96 bp, Artificial
  Control Probe
uuuuacgacttttacttcctctaggcgtatgcggattgggctcctcactaaaccatccaagcgtatgcggattgggctaaagggcagggacttaat

SEQ ID NO: 2 - DNA, 18 bp, Artificial
  Surface Probe
caaggtttccgtaggtga

SEQ ID NO: 3 - DNA, 20 bp, Artificial
  DV59 Probe
ctagaggaagtaaaagtcgt

SEQ ID NO: 4 - DNA, 18 bp, Artificial
  DV150 Probe
ttggatggtttagtgagg

SEQ ID NO: 5 - DNA, 18 bp, Artificial
  DV195 Probe
attaagtccctgcccttt

SEQ ID NO: 6 - DNA, 23 bp, Artificial
  Capture Probe
uuuuutcacctacggaaaccttg

(19) United States
(12) Patent Application Publication
(10) Pub. No.: US 2022/0170083 A1
(43) Pub. Date: Jun. 2, 2022

(54) RNA INTEGRITY ANALYSIS IN A BIOLOGICAL SAMPLE

(71) Applicant: 10x Genomics, Inc., Pleasanton, CA (US)

(72) Inventors: Fatme Khaled, Stockholm (SE); Linda Kvastad, Huddinge (SE)

(21) Appl. No.: 17/670,147

(22) Filed: Feb. 11, 2022

Related U.S. Application Data
(63) Continuation of application No. PCT/US2021/033599, filed on May 21, 2021.
(60) Provisional application No. 63/029,151, filed on May 22, 2020.

(51) Int. Cl. C12Q 1/6841

(52) U.S. Cl. CPC C12Q 1/6841

(57) ABSTRACT

Described herein is an assay capable of investigating nucleic acid integrity in a biological sample.

### CROSS-REFERENCE TO RELATED APPLICATIONS

Pursuant to 35 U.S.C. § 119(e), this application is a continuation of International Application PCT/US2021/033599, with an international filing date of May 21, 2021, which claims the benefit of U.S. Provisional Patent Applications No. 63/029,151, filed on May 22, 2020, the contents of which are hereby incorporated by reference herein in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Biological samples for spatial analysis are quality controlled by extraction of RNA followed by an RNA integrity analysis (e.g., RNA Integrity Number (RIN)). Spatial fragment distribution value (DV) is another method of measuring RNA integrity (e.g., degradation) in a biological sample, including fixed biological samples, and can also identify spatial patterns of degradation within a biological sample.

### SUMMARY

A fundamental tool in anatomical pathology for disease diagnosis is preserving tissues in the form of formalin-fixed paraffin-embedded (FFPE) samples. A major advantage of this type of sample is its ability to maintain the morphology and structure of cells within a tissue sample, which is the basis of disease diagnosis and biomarker detection. This advantage has made FFPE specimens a popular approach for long-term preservation of biological samples (e.g., tissue sections). However, since the crosslinks introduced through the fixation significantly affect the integrity of the nucleic acids within, their use is limited, especially in studies that involve gene expression analysis. Therefore, developing a workflow that enables determination of nucleic acid integrity (e.g., quality) in fixed (e.g., FFPE) biological samples will have a positive impact on both the research community and pathology departments.

Numerous studies have investigated and evaluated the integrity of fixed biological samples and their genomic content. There remains a need to provide estimations of FFPE nucleic acids integrity as a function of their spatial distribution.

Despite potential drawbacks linked to fixed biological samples, several studies have shown that nucleic acids (e.g., RNA) derived from FFPE samples can still be used to generate transcriptome information comparable to fresh frozen biological samples. However, since RNA integrity varies in different fixed biological samples (e.g., FFPE), not all fixed biological samples can generate usable high-quality data. Thus, performing gene expression analysis on fixed biological samples with high degradation levels are most likely to fail in providing interpretable results. In order to avoid wasting reagents and time associated with expression analysis methods, a quality control assay can determine whether an analysis method will provide accurate data from a biological sample.

For example, spatially determining RNA integrity in sub-areas of the tissue, including regions of interest, can facilitate the examination of fixed biological samples and ensure that sub-areas of the biological sample, including a region of interest, contain nucleic acids of sufficient quality to provide data for downstream analyses, including spatial transcriptomics. Thus, provided herein are methods for assessing the integrity of nucleic acids obtained from a fixed biological sample (e.g., a formalin-fixed paraffin-embedded biological sample).

Thus provided herein are methods of determining the presence of RNA of sufficient integrity suitable for downstream applications in a fixed biological sample, the method including: (a) generating a spatial fragment distribution value (DV) number of the fixed biological sample; (b) generating an RNA integrity number (RIN) score of the FFPE biological sample; and (c) using the generated spatial fragment distribution value (DV) number of step (a), and the RIN score of the fixed biological sample of step (b), to determine the presence of RNA of sufficient integrity suitable for downstream applications in the fixed biological sample.

In some embodiments, determining the spatial fragment DV number includes: (a) contacting the fixed biological sample with a substrate including a plurality of capture probes, where a capture probe of the plurality of capture probes includes a capture domain that is capable of binding specifically to a ribosomal RNA (rRNA) from the fixed biological sample; (b) de-crosslinking one or more crosslinks in the fixed biological sample; (c) permeabilizing the fixed biological sample under conditions sufficient to allow the rRNA to bind specifically to the capture domain; (d) extending an end of the capture probe using the rRNA specifically bound to the capture domain as a template, thereby generating an extended capture probe; (e) contacting a first detectable probe to the extended capture probe, where the first detectable probe includes (i) a sequence that corresponds to a first sequence present in a 3′ region of the rRNA, and (ii) a first detectable label; and (f) detecting a location of the first detectable label on the substrate, thereby determining a spatial fragment DV number of the first detectable label.

In some embodiments, extending the end of the capture probe includes extending a 3′ end of the capture probe. In some embodiments, extending the 3′ end of the capture probe includes generating a single-stranded cDNA.

In some embodiments, the method includes, before step (c), a step of staining and imaging the fixed biological sample. In some embodiments, the fixed biological sample is stained with hematoxylin and eosin.

In some embodiments, the rRNA is an 18S rRNA. In some embodiments, the rRNA is a 28S rRNA.

In some embodiments, the de-crosslinking step includes heating the fixed biological sample. In some embodiments, the de-crosslinking step includes the performance of a chemical reaction. In some embodiments, the de-crosslinking step includes the use of an enzyme. In some embodiments, the de-crosslinking step includes the use of TE buffer. In some embodiments, the TE buffer has a temperature of about 65° C. to about 75° C., and is contacted with the fixed biological sample for about 30 minutes to about 90 minutes.

In some embodiments, the step of extending the end of the capture probe is performed in the presence of actinomycin D.

In some embodiments, the method includes treating the fixed biological sample with an RNase after step (d). In some embodiments, the RNase is RNase H. In some embodiments, the step of permeabilizing the fixed biological sample includes the use of a protease. In some embodiments, the protease is pepsin or proteinase K.

In some embodiments, the fixed biological sample is removed after the extending in step (d).

In some embodiments, the method includes contacting a second detectable probe to the extended capture probe, where the second detectable probe includes (i) a sequence that corresponds to a second sequence in the rRNA that is positioned 5′ relative to the first sequence in the rRNA, and (ii) a second detectable label; and detecting a location of the second detectable label on the substrate, thereby determining a spatial fragment DV number of the second detectable label.

In some embodiments, the method includes contacting a third detectable probe to the extended capture probe, where the third detectable probe includes (i) a sequence that corresponds to a third sequence in the rRNA that is positioned 5′ relative to the second sequence in the rRNA, and (ii) a third detectable label; and detecting a location of the third detectable label on the substrate, thereby determining a spatial fragment DV number of the third detectable label.

In some embodiments, the first detectable label, the second detectable label, and the third detectable label is a fluorophore. In some embodiments, first detectable label, the second detectable label, and the third detectable label are different. In some embodiments, the first detectable label, the second detectable label, and the third detectable label are the same. In some embodiments, the method includes, a step of disassociating and removing the first detectable probe from the extended capture probe prior to contacting the substrate with the second detectable probe. In some embodiments, the method includes, a step of disassociating and removing the second detectable probe from the extended capture probe prior to contacting the substrate with the third detectable probe.

In some embodiments, the first detectable probe detects a short extended capture probe. In some embodiments, the short extended capture probe includes an extended capture probe of about 60 nucleotides or less from the 3′ end of the captured analyte. In some embodiments, the second detectable probe detects a mid-length extended capture probe. In some embodiments, the mid-length extended capture probe includes an extended capture probe from at least about 120 nucleotides to about 180 nucleotides from the 3′ end of the captured analyte. In some embodiments, the third detectable probe detects a long extended capture probe. In some embodiments, the long extended capture probe includes an extended capture probe from at least about 180 nucleotides to about 220 nucleotides from the 3′ end of the captured analyte.

In some embodiments, the spatial fragment DV number of the FFPE biological sample includes a number between 1 and 100. In some embodiments, the spatial fragment DV number of the long extended capture probe includes 60 or greater. In some embodiments, the spatial fragment DV number of 60 or greater of the long extended capture probe or greater is indicative of RNA of sufficient integrity suitable for downstream applications.

In some embodiments, generating the RIN score for the fixed biological sample includes a score between 1 and 10. In some embodiments, the RIN score of 7 or greater is indicative of RNA of sufficient integrity suitable for downstream applications. In some embodiments, the fixed biological sample includes the spatial fragment DV number of the long extended capture probe of less than 60 and the RIN score of less than 7. In some embodiments, the fixed biological sample includes the spatial fragment DV number of the long extended capture probe of 60 or greater and the RIN score of less than 7.

In some embodiments, a downstream application includes spatial transcriptomics.

In some embodiments, the fixed sample is a formalin-fixed paraffin-embedded sample biological sample, a PFA fixed biological sample, or an acetone fixed biological sample. In some embodiments, the fixed biological sample is an FFPE tissue section, a PFA tissue section, or an acetone fixed tissue section. In some embodiments, the fixed biological sample is a tumor sample.

Also provided herein, are methods for generating a spatial fragment distribution value (DV) heat map of a fixed biological sample, the method including: (a) contacting the fixed biological sample with a substrate including a plurality of capture probes, where a capture probe of the plurality of capture probes includes a capture domain that is capable of binding specifically to a ribosomal RNA (rRNA) from the fixed biological sample; (b) de-crosslinking one or more crosslinks in the fixed biological sample; (c) permeabilizing the fixed biological sample under conditions sufficient to allow the rRNA to bind specifically to the capture domain; (d) extending an end of the capture probe using the rRNA specifically bound to the capture domain as a template, thereby generating an extended capture probe; (e) contacting a first detectable probe to the extended capture probe, where the first detectable probe includes (i) a sequence that corresponds to a first sequence present in a 3′ region of the rRNA, and (ii) a first detectable label; (f) detecting a location of the first detectable label on the substrate; (g) contacting a second detectable probe to the extended capture probe, where the second detectable probe includes (i) a sequence that corresponds to a second sequence in the rRNA that is positioned 5′ relative to the first sequence in the rRNA, and (ii) a second detectable label; (h) detecting a location of the second detectable label on the substrate; and (i) comparing the location of the first detectable label to the location of the second detectable label on the substrate, thereby generating the spatial fragment DV heat map of the fixed biological sample.

In some embodiments, extending the end of the capture probe includes extending a 3′ end of the capture probe. In some embodiments, extending the 3′ end of the capture probe includes generating a single-stranded cDNA.

In some embodiments, the method includes, before step (c), a step of staining and imaging the fixed biological sample. In some embodiments, the fixed biological sample is stained with hematoxylin and eosin.

In some embodiments, the rRNA is an 18S rRNA or a 28S rRNA.

In some embodiments, the de-crosslinking step includes heating the fixed biological sample. In some embodiments, the de-crosslinking step includes the performance of a chemical reaction. In some embodiments, the de-crosslinking step includes the use of an enzyme. In some embodiments, the de-crosslinking step includes the use of TE buffer. In some embodiments, the TE buffer has a pH of about 7.5 to about 8.5. In some embodiments, the TE buffer has a temperature of about 65° C. to about 75° C., and is contacted with the fixed biological sample for about 30 minutes to about 90 minutes.

In some embodiments, the step of extending the end of the capture probe is performed in the presence of actinomycin D.

In some embodiments, the method includes treating the fixed biological sample with an RNase after step (d). In some embodiments, the RNase is RNase H.

In some embodiments, the step of permeabilizing the FFPE biological sample includes the use of a protease. In some embodiments, the protease is pepsin or proteinase K.

In some embodiments, the fixed biological sample is removed after the extending in step (d).

In some embodiments, one or both of the first detectable label and the second detectable label is a fluorophore. In some embodiments, step (f) and/or step (h) includes detecting fluorescence of the first and/or second detectable label. In some embodiments, the first detectable label and the second detectable label are different. In some embodiments, the first detectable label and the second detectable label are the same. In some embodiments, the method includes, between steps (g) and (h), a step of disassociating and removing the first detectable probe from the extended capture probe. In some embodiments, the method includes, between steps (h) and (i), a step of disassociating and removing the second detectable probe from the extended capture probe.

In some embodiments, the method includes contacting a third detectable probe to the extended capture probe, where the third detectable probe includes (i) a sequence that corresponds to a third sequence in the rRNA that is positioned 5′ relative to the second sequence in the rRNA, and (ii) a third detectable label. In some embodiments, the method includes detecting a location of the third detectable label on the substrate. In some embodiments, the first detectable label, the second detectable label, and the third detectable label are different. In some embodiments, the first detectable label, the second detectable label, and the third detectable label are the same.

In some embodiments, step (i) includes comparing the location of the first detectable label on the substrate, the second detectable label on the substrate, and the location of the third detectable label on the substrate, thereby generating the spatial fragment DV heat map of the FFPE biological sample.

In some embodiments, the fixed biological sample is a formalin-fixed paraffin-embedded biological sample, a PFA fixed biological sample, or an acetone fixed biological sample. In some embodiments, the fixed biological sample is an FFPE tissue section, a PFA tissue section, or an acetone fixed tissue section. In some embodiments, the fixed biological sample is a tumor sample.

In some embodiments, the spatial fragment DV heat map identifies a region of interest in the fixed biological sample.

In some embodiments, the method includes determining a spatial fragment DV number for the fixed biological sample.

In some embodiments, the spatial fragment DV number is an indication of RNA degradation in the fixed biological sample.

In some embodiments, the method includes sequencing the extended capture probe, where the extended capture probe includes a spatial barcode.

In some embodiments, the method includes (a) contacting the fixed biological sample with a substrate including a plurality of capture probes, where a capture probe of the plurality of capture probes includes a capture domain that is capable of binding specifically to a ribosomal RNA (rRNA) from the FFPE biological sample; (b) staining and imaging the fixed biological sample; (c) de-crosslinking one or more crosslinks in the fixed biological sample; (d) extending an end of the capture probe using the rRNA specifically bound to the capture domain as a template, thereby generating an extended capture probe; (e) contacting a first detectable probe to the extended capture probe, where the first detectable probe includes (i) a sequence that corresponds to a first sequence present in a 3′ region of the rRNA, and (ii) a first detectable label; (f) detecting a location of the first detectable probe on the substrate; (g) contacting a second detectable probe to the extended capture probe, where the second detectable probe includes (i) a sequence that corresponds to a second sequence in the rRNA that is positioned 5′ relative to the first sequence in the rRNA, and (ii) a second detectable label; (h) detecting a location of the second detectable label on the substrate; and (i) comparing a stained image of the fixed biological sample and the location of the first detectable label and the second label on the substrate, thereby generating the spatial fragment DV heat map of the fixed biological sample.

Also provided herein are arrays including a plurality of capture probes, where a capture probe of the plurality of capture probes includes a capture domain that is capable of specifically binding to a sequence present in an 18S ribosomal RNA.

In some embodiments, the capture probe includes a spatial barcode. In some embodiments, the capture probe includes one or more functional domains, a cleavage domain, a unique molecular identifier, and combinations thereof.

Also provided herein are kits including: any of the array of any one of claims 80-82; a first detectable probe, where the first detectable probe includes (i) a sequence that corresponds to a first sequence present in a 3′ region of the rRNA, and (ii) a first detectable label; a second detectable probe, where the second detectable probe includes (i) a sequence that corresponds to a second sequence in the rRNA that is positioned 5′ relative to the first sequence in the rRNA, and (ii) a second detectable label.

In some embodiments, one or both of the first detectable label and the second detectable label is a fluorophore. In some embodiments, the first detectable label and the second detectable label are different. In some embodiments, the first detectable label and the third detectable label are the same.

In some embodiments, the kit includes a third detectable probe, where the third detectable probe includes (i) a sequence that corresponds to a third sequence in the rRNA that is positioned 5′ relative to the second sequence in the rRNA, and (ii) a third detectable label. In some embodiments, the third detectable label is a fluorophore.

In some embodiments, the first detectable label, the second detectable label, and the third detectable label are different. In some embodiments, the first detectable label, the second detectable label, and the third detectable label are the same.

In some embodiments, the kit includes one or more permeabilization reagents. In some embodiments, the one or more permeabilization reagents includes TE buffer.

In some embodiments, the kit includes a protease. In some embodiments, the kit includes a nuclease. In some embodiments, the kit includes a reverse transcriptase.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 shows an exemplary workflow for spatial distribution fragment value analysis in a biological sample.

FIG. 3A is an exemplary graph showing the hybridization efficiency of different DV probes and a surface probe at 50° C.

FIG. 3B is an exemplary graph showing the hybridization efficiency of different DV probes and a surface probe at 60° C.

FIG. 3C is an exemplary graph showing the hybridization efficiency of different DV probes and a surface probe at 70° C.

FIG. 4A is an exemplary graph showing RIN values from a fresh frozen biological sample.

FIG. 4B is an exemplary graph showing RIN values from a fresh frozen biological sample.

FIG. 5A shows an exemplary graph showing a RIN value and spatial fragment DV numbers from a formalin-fixed paraffin-embedded biological sample.

FIG. 5B shows an exemplary graph showing a RIN value and spatial fragment DV numbers from a formalin-fixed paraffin-embedded biological sample.

FIG. 5C shows an exemplary graph showing a RIN value and spatial fragment DV numbers from a formalin-fixed paraffin-embedded biological sample.

FIG. 5D shows an exemplary graph showing a RIN value and spatial fragment DV numbers from a formalin-fixed paraffin-embedded biological sample.

FIG. 5E shows an exemplary graph showing a RIN value and spatial fragment DV numbers from a formalin-fixed paraffin-embedded biological sample.

FIG. 6A is an exemplary H&E stained biological sample.

FIG. 6B is an exemplary spatial fragment DV map of a biological sample.

FIG. 6C is an exemplary spatial fragment DV map of a biological sample.

FIG. 6D is an exemplary control for a spatial fragment DV map of a biological sample.

FIG. 7A is an exemplary H&E stained biological sample.

FIG. 7B is an exemplary spatial fragment DV map of a biological sample.

FIG. 7C is an exemplary spatial fragment DV map of a biological sample.

FIG. 7D is an exemplary control for a spatial fragment DV map of a biological sample.

FIG. 8A is an exemplary H&E stained biological sample.

FIG. 8B is an exemplary spatial fragment DV map of a biological sample.

FIG. 8C are exemplary close-up images of the H&E stained biological sample shown in FIG. 8A in three separate regions denoted 1, 2, and 3.

FIG. 8D are exemplary close-up images of the spatial fragment DV map of the biological sample shown in FIG. 8B in three separate regions denoted 1, 2, and 3.

FIG. 9A shows an exemplary a positive control in a spatial fragment DV assay for a reference sample.

FIG. 9B shows an exemplary negative control in a spatial fragment DV assay for no biological sample.

FIG. 10A-B shows an exemplary side-by-side comparison of a graph with a RIN value and spatial fragment DV numbers from a formalin-fixed paraffin-embedded biological sample.

FIG. 11A-B shows an exemplary side-by-side comparison of a graph with a RIN value and spatial fragment DV numbers from a formalin-fixed paraffin-embedded biological sample.

### DETAILED DESCRIPTION

A fundamental tool in anatomical pathology for disease diagnosis is preserving tissues in the form of formalin-fixed paraffin-embedded (FFPE) samples. A major advantage of this type of sample is its ability to maintain the morphology and structure of cells within a tissue sample, which is the basis of disease diagnosis and biomarker detection. This advantage has made FFPE specimens a popular approach for long-term preservation of biological samples (e.g., tissue sections). However, since the crosslinks introduced through the fixation significantly affect the integrity of the nucleic acids within, their use is limited, especially in studies that involve gene expression analysis. Therefore, developing a workflow that enables determination of nucleic acid integrity (e.g., quality) in fixed (e.g., FFPE) biological samples will have a positive impact on both the research community and pathology departments.

Numerous studies have investigated and evaluated the integrity of fixed biological samples and their genomic content. There remains a need to provide estimations of FFPE nucleic acids integrity as a function of their spatial distribution.

Despite potential drawbacks linked to fixed biological samples, several studies have shown that nucleic acids (e.g., RNA) derived from FFPE samples can still be used to generate transcriptome information comparable to fresh frozen biological samples. However, since RNA integrity varies in different fixed biological samples (e.g., FFPE), not all fixed biological samples can generate usable high-quality data. Thus, performing gene expression analysis on fixed biological samples with high degradation levels are most likely to fail in providing interpretable results. In order to avoid wasting reagents and time associated with expression analysis methods, a quality control assay can determine whether an analysis method will provide accurate data from a biological sample.

For example, spatially determining RNA integrity in sub-areas of the tissue, including regions of interest, can facilitate the examination of fixed biological samples and ensure that sub-areas of the biological sample, including a region of interest, contain nucleic acids of sufficient quality to provide data for downstream analyses, including spatial transcriptomics.

Provided herein are methods for assessing the integrity of nucleic acids obtained from a fixed biological sample (e.g., a formalin-fixed paraffin-embedded biological sample). Some embodiments of any of the methods described herein include determining a spatial fragment distribution number (e.g., value) for a fixed biological sample. Some embodiments of any of the methods described herein can include the generation of a spatial fragment distribution heat map. Some embodiments of any of the methods described herein can include the use of one or more detectable probes for a ribosomal RNA (e.g., 18S ribosomal RNA). In some embodiments of any of the methods described herein include the use of one or more detectable probes for 28S ribosomal RNA.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

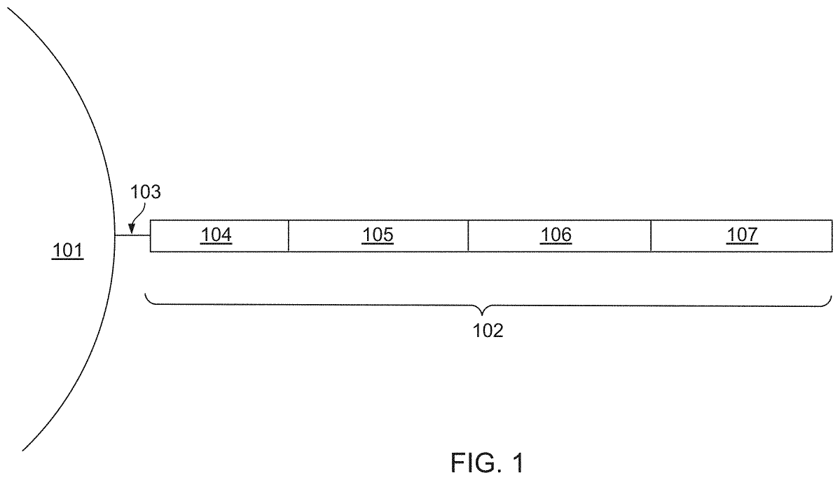

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte.

In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No.

2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 August 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Determining RNA Integrity in FFPE Samples

Spatially determining RNA integrity in a biological sample, sub-areas of the biological sample, or regions of interest in a biological sample can facilitate the examination of fixed biological samples and ensure that sub-areas of the biological sample, including regions of interest, contain nucleic acids of sufficient integrity (e.g., quality) to provide data for downstream analyses, including spatial transcriptomics.

Provided herein are methods, compositions, and kits for assessing the integrity (e.g., quality) of nucleic acids from a biological sample. In some embodiments, the biological sample is a fixed biological sample (e.g., formalin-fixed paraffin-embedded biological sample (FFPE), paraformaldehyde (PFA), acetone, etc.). In some embodiments, assessing the integrity of the nucleic acids includes determining a spatial fragment distribution number (e.g., value). In some embodiments, assessing the integrity of nucleic acids from a biological sample includes generating a spatial fragment distribution heat map. In some embodiments, assessing the integrity of nucleic acids in a biological sample include one or more detectable probes for a ribosomal RNA (rRNA). In some embodiments, the one or more detectable probes are probes for 18S rRNA. In some embodiments, the one or more detectable probes are for 28S ribosomal RNA.

As used herein, “spatial fragment distribution value (DV)” refers to a measurement of nucleic acid integrity in a biological sample (e.g., FFPE biological sample) obtained from a spatial fragment DV assay (Zhao, Y., et al., Robustness of RNA sequencing on older formalin-fixed paraffin-embedded tissue from high-grade ovarian serous adenocarcinomas. PloS One, 14: e0216050 (2019)). A spatial fragment DV can be represented in multiple ways. For example, a spatial fragment DV can be represented as a “spatial fragment DV number” from 1 to 100. The spatial fragment DV number is generated by detecting one or more detectable probes specifically bound to an extended capture probe (e.g., an extended capture probe generated by using rRNA as a template), or a complement thereof. The one or more detectable probes can be designed to detect different locations of the extended capture probe, or complement thereof, which can represent the integrity of the nucleic acid in the biological sample. A spatial fragment DV can also be represented as a “spatial fragment DV heat map” that can indicate a spatial fragment DV at one or more locations in the biological sample. In some embodiments, a spatial fragment DV heat map can be generated by detecting one or more detectable probes specifically bound to an extended capture probe, imaging the biological sample (e.g., FFPE, PFA, acetone fixed biological sample), disassociating one or more detectable probes, and repeating the process. The images obtained by detecting the one or more detectable probes (e.g., a first detectable probe, a second detectable probe, or more) can be compared and viewed as a spatial fragment (DV) heat map.

Provided herein are methods for generating a spatial fragment distribution value (DV) heat map of a formalin-fixed paraffin-embedded (FFPE) biological sample, the method including contacting the FFPE biological sample with a substrate including a plurality of capture probes, where a capture probe of the plurality of capture probes includes a capture domain that is capable of binding specifically to a ribosomal RNA (rRNA) from the FFPE biological sample, de-crosslinking one or more formaldehyde crosslinks in the FFPE biological sample, permeabilizing the FFPE biological sample under conditions sufficient to allow the rRNA to bind specifically to the capture domain, extending an end of the capture probe using the rRNA specifically bound to the capture domain as a template, thereby generating an extended capture probe, contacting a first detectable probe to the extended capture probe, where the first detectable probe includes a sequence that corresponds to a first sequence present in a 3′ region of the rRNA and a first detectable label, detecting a location of the first detectable label on the substrate, contacting a second detectable probe to the extended capture probe, where the second detectable probe includes a sequence that corresponds to a second sequence in the rRNA that is positioned 5′ relative to the first sequence in the rRNA and a second detectable label, detecting a location of the second detectable label on the substrate, and comparing the location of the first detectable label to the location of the second detectable label on the substrate, thereby generating the spatial fragment DV heat map of the FFPE biological sample for evaluating the RNA integrity of the biological sample. However, the methods and compositions described herein could be equally applied to other tissue or cell fixation methods including, but not limited to, PFA, acetone, and methanol. It is contemplated that as long as the fixative does not hinder downstream enzymatic reactions, for example, reverse transcription reactions, that the fixative does not hinder removal of the tissue from the substrate, and the fixative does not leave residual auto-fluorescence (e.g., which may interfere as background for subsequent target fluorescence detection of detection probe emissions) on the substrate the fixative would be compatible with the disclosed methods for determining RNA integrity of a biological sample.

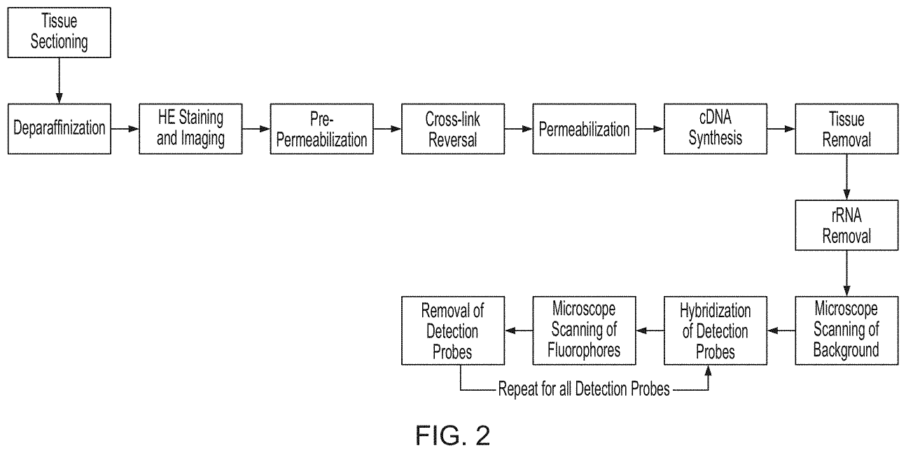

Generally the methods disclosed herein include the exemplary workflow shown in FIG. 2 and more fully described in the Examples. The workflow shown in FIG. 2 includes collecting a biological sample (e.g., tissue sectioning), deparaffinization, staining (e.g., H&E staining), pre-permeabilization, reversal of crosslinks, permeabilization (e.g., by any of the permeabilization methods described herein), cDNA synthesis (e.g., first strand cDNA synthesis), removal of the tissue, removal of nucleic acids, and a series of sequential hybridizations with detectable probes (e.g., fluorescently labeled), where the detectable probes are removed between each subsequent hybridization round.

In some embodiments of generating a spatial fragment distribution value (DV) heat map from a fixed sample include contacting the fixed sample with a substrate including a plurality of capture probes, wherein a capture probe of the plurality of capture probes comprises a capture domain that is capable of binding specifically to a ribosomal RNA (rRNA) from the fixed biological sample, staining (e.g., H&E stain) and imaging the fixed biological sample, de-crosslinking one or more formaldehyde crosslinks in the fixed biological sample, extending an end of the capture probe using the rRNA specifically bound to the capture domain as a template, thereby generating an extended capture probe, contacting a first detectable probe to the extended capture probe, where the first detectable probe includes a sequence that corresponds to a first sequence present in a 3′ region of the rRNA and a first detectable label, detecting a location of the first detectable probe on the substrate, contacting a second detectable probe to the extended capture probe, where the second detectable probe includes a sequence that corresponds to a second sequence in the rRNA that is positioned 5′ relative to the first sequence in the rRNA and a second detectable label, detecting a location of the second detectable label on the substrate, and comparing a stained image of the fixed biological sample and the location of the first detectable label and the second label on the substrate, thereby generating the spatial fragment DV heat map of the fixed biological sample.

Any suitable fixative or fixation methods (e.g., embedding materials) can be used, including for example, ethanol, methanol, paraformaldehyde or formaldehyde. In some embodiments, the biological sample is an FFPE biological sample. For example, the biological sample can be fixed in a suitable fixative, typically formalin, and embedded in melted paraffin wax. The wax block can be cut on a microtome to yield a thin slice of paraffin containing the biological sample. The biological sample can be applied to a substrate, air dried, and heated to cause the specimen to adhere to the glass slide. Residual paraffin can be dissolved with a suitable solvent, typically xylene, toluene, or others. These deparaffinizing solvents can be removed with washing and/or dehydrating reagents prior to staining. Sliced biological samples can be prepared from frozen specimens, fixed briefly in 10% formalin, and infused with a dehydrating reagent.

In some embodiments, the paraffin-embedding material can be removed (e.g., deparaffinization) from the biological sample (e.g., tissue section) by incubating the biological sample in an appropriate solvent (e.g., xylene), followed by a series of rinses (e.g., ethanol of varying concentrations), and rehydration in water. In some embodiments, the biological sample can be dried following deparaffinization. In some embodiments, after the step of drying the biological sample, the biological sample can be stained (e.g., H&E stain, any of the variety of stains described herein). In some embodiments, after staining the biological sample, the sample can be imaged.

In some embodiments, the biological sample is a PFA fixed biological sample. In some embodiments, the biological sample is an acetone fixed biological sample.

After an FFPE biological sample has undergone deparaffinization, the FFPE biological sample can be further processed. For example, FFPE biological samples can be treated to remove formaldehyde-induced crosslinks (e.g., decrosslinking). In some embodiments, de-crosslinking the formaldehyde-induced crosslinks in the FFPE biological sample can include treating the sample with heat. In some embodiments, decrosslinking the formaldehyde-induced crosslinks can include performing a chemical reaction. In some embodiments, decrosslinking the formaldehyde-induced crosslinks, can include treating the sample with a permeabilization reagent. In some embodiments, decrosslinking the formaldehyde-induced crosslinks can include heat, a chemical reaction, and/or permeabilization reagents.

In some embodiments, decrosslinking formaldehyde-induced crosslinks can be performed in the presence of a buffer. For example, the buffer can be Tris-EDTA (TE) buffer. In some embodiments, the TE buffer has a pH of about 7.0 to about 9.0, about 7.1 to about 8.9, about 7.2 to about 8.8, about 7.3 to about 8.7, about 7.4 to about 8.6, about 7.5 to about 8.5, about 7.6 to about 8.4, about 7.7 to about 8.3, about 7.8 to about 8.2, about 7.9 to about 8.1, or about 8.0.

In some embodiments, the TE buffer has a temperature of about 60° C. to about 80° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78, about 79° C., or about 80° C.

In some embodiments, the fixed biological sample can be contacted with TE buffer for about 10 minutes to about 200 minutes, about 10 minutes to about 190 minutes, about 10 minutes to about 180 minutes, about 10 minutes to about 170 minutes, about 10 minutes to about 160 minutes, about 10 minutes to about 160 minutes, about 10 minutes to about 150 minutes, about 10 minutes to about 140 minutes, about 10 minutes to about 130 minutes, about 10 minutes to about 120 minutes, about 10 minutes to about 110 minutes, about 10 minutes to about 100 minutes, about 10 minutes to about 90 minutes, about 10 minutes to about 80 minutes, about 10 minutes to about 70 minutes, about 10 minutes to about 60 minutes, about 10 minutes to about 50 minutes, about 10 minutes to about 40 minutes, about 10 minutes to about 30 minutes, or about 10 minutes to about 20 minutes.

In some embodiments, the fixed biological sample can be contacted with TE buffer that has a temperature of about 65° C. to about 75° C., and is contacted with the fixed biological sample for about 30 minutes to about 90 minutes. In some embodiments, the TE buffer can have a temperature of about 70° C., a pH of about 8.0, and can be contacted with the fixed biological sample for about 60 minutes.

After decrosslinking the formaldehyde crosslinks (e.g., decrosslinking) in the fixed biological sample (e.g., FFPE tissue section, PFA tissue section, acetone tissue section), the biological sample can be permeabilized (e.g., permeabilized by any of the variety of methods described herein). In some embodiments, the fixed biological sample can be permeabilized with a protease. In some embodiments, the protease can be pepsin. In some embodiments, the protease can be proteinase K. In some embodiments, the protease can be pepsin and proteinase K. In some embodiments, the fixed biological sample can be permeabilized with a protease for about 10 minutes to about 60 minutes.

In some embodiments, the thickness of the biological sample (e.g., tissue section), for use in the methods described herein may be dependent on the method used to prepare the sample and the physical characteristics of the tissue. Thus, any suitable section thickness can be used. In some embodiments, the thickness of the biological sample section will be at least 0.1 μm, further preferably at least 0.2, 0.3, 0.4, 0.5, 0.7, 1.0, 1.5, 2, 3, 4, 5, 6, 7, 8, 9, or 10 μm. In some embodiments the thickness of the biological sample section is at least 10, 11, 12, 13, 14, 15, 20, or 30 μm. In some embodiments, the thickness of the biological sample is 5-12 μm.

The analyte in the nucleic acid integrity assay (e.g., spatial fragment DV assay) refers to a nucleic acid present in the biological sample. In some embodiments, the analyte is RNA.

In some embodiments, the analyte is a coding RNA. In some embodiments, the analyte is a non-coding RNA. In some embodiments, the RNA is messenger RNA (mRNA) or ribosomal RNA (rRNA). In some embodiments, the RNA is double-stranded RNA. In some embodiments, the RNA is single-stranded RNA. In some embodiments, the RNA is a circular RNA. It is contemplated that as long as an RNA is at least 200 nt long and is abundant in a cell it could serve as a template for measuring RNA integrity.

In some embodiments, a fixed biological sample is contacted with a substrate including a plurality of capture probes (e.g., any of the capture probes described herein). In some embodiments, the capture probes include a capture domain. In some embodiments, the capture domain is substantially complementary to an analyte having a nucleic acid sequence.

In some embodiments, the capture domain is substantially complementary to an RNA. In some embodiments, the capture domain is substantially complementary to ribosomal RNA. In some embodiments, the capture domain is substantially complementary to 18S rRNA. In some embodiments, the capture domain is substantially complementary to 28S rRNA. In some embodiments, the capture domain includes a sequence with SEQ ID NO: 6.

In some embodiments, the capture domain includes a sequence that is at least 70%, at least 75%, at least 80%, at least 85%, at least 90%, at least 95%, at least 96%, at least 97%, at least 98%, or at least 99% complementary to a nucleic acid (e.g., 18S rRNA), or a portion thereof. In some embodiments, the capture domain includes a sequence that is perfectly complementary (e.g., is 100% complementary) to a nucleic acid (e.g., 18S rRNA). In some embodiments, the capture domain is capable of capturing nucleic acid (e.g., rRNA) from biological samples obtained from different species. For example, rRNA is highly conserved amongst many species and the capture domain can be designed to capture rRNA from biological samples obtained from different species.

In some embodiments of the nucleic acid integrity assay methods described herein, after the capture probe captures an analyte (e.g., rRNA, 18S rRNA), a reverse transcription reaction is performed thereby generating an extended capture probe (e.g., single stranded cDNA sequence complementary to the captured analyte, e.g., 18S rRNA). Any suitable reverse transcriptase can be used to generate the single-stranded cDNA, including any reverse transcriptases described herein. In some embodiments, extending the end of the capture probe is performed in the presence of actinomycin D.

In some embodiments, the biological sample is treated with a nuclease after the step of extending the capture probe. In some embodiments, the nuclease is an RNase. A non-limiting example of an RNase is RNase H. In some embodiments, the RNase degrades RNA present in the biological sample. In some embodiments, the RNase degrades the captured rRNA hybridized to the extended capture probe (e.g., single-stranded cDNA generated by reverse transcription). In some embodiments after reverse transcription (e.g., single-stranded cDNA synthesis) the biological sample is removed. For example, the biological sample can be treated with one or more permeabilization reagents to remove the biological sample. In some embodiments, the one or more permeabilization reagents include TE buffer and one or more proteases as described herein. In some embodiments, after reverse transcription, the biological sample is not removed.

After treating the biological sample with a nuclease (e.g., RNase) and/or removal of the biological sample, one or more detectable probes can be contacted with the substrate including the capture probes (e.g., array). In some embodiments, the detectable probes are labeled where the detection of the label represents hybridization to the extended capture probe (e.g., single-stranded cDNA), or a complement thereof. The detectable label can be any of the detectable labels described herein (e.g., Cy3, Cy5, etc.). In some embodiments, a first detectable probe is contacted with the array where the first detectable probe hybridizes to a portion of the extended capture probe (e.g., single-stranded cDNA), or a complement thereof. In some embodiments, the first detectable probe is detected by microscope scanning for the fluorophores. In some embodiments, the first detectable probe is disassociated (e.g., dehybridized and washed) from the array. The process of contacting the array with one or more detectable probes (e.g., a first detectable probe, a second detectable probe, a third detectable probe, a fourth detectable probe, or more) followed by disassociation, can be repeated 2, 3, 4, or more times. In some embodiments, one or more second detectable probes are contacted with the array where a detectable probe hybridizes to a portion of the extended capture probe (e.g., single-stranded cDNA), or a complement thereof. In some embodiments, the one or more second detectable probes are detected by microscope scanning for the fluorophores. In some embodiments, the one or more second detectable probes are disassociated (e.g., dehybridized and washed) from the array. In some embodiments, one or more third detectable probes are contacted with the array where a third detectable probe hybridizes to a portion of the extended captured probe (e.g., single-stranded cDNA), or a complement thereof. In some embodiments, the one or more third detectable probes are detected by microscope scanning for the fluorophores. In some embodiments, the one or more third detectable probes are disassociated (e.g., dehybridized and washed) from the array. In some embodiments, the one or more first detectable probes, the one or more second detectable probes, and the one or more third detectable probes can have a sequence, for example, the one or more first detectable probes can have a sequence comprising SEQ ID NO: 3, the one or more second detectable labels can have a sequence comprising SEQ ID NO: 4, and the one or more third detectable labels can have a sequence comprising SEQ ID NO: 5.

In some embodiments of the RNA integrity assay methods described herein, a spatial fragment distribution value (DV) heat map can be generated by detecting a first detectable probe, a second detectable probe, and a third detectable probe. In some embodiments the first, second, and/or third detectable probes can be designed to assess the integrity of the RNA present in a biological sample.

In some embodiments, a detectable probe can be from about 10 nucleotides long to about 30 nucleotides long. In some embodiments, a detectable probe can be from about 15 nucleotides long to about 25 nucleotides long. In some embodiments, a detectable probe can be about 20 nucleotides long. In some embodiments, a detectable probe can be from about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides long.

In some embodiments, the first detectable probe, the second detectable probe, and the third detectable probe include a detectable label (e.g., any of the detectable labels described herein). In some embodiments, the detectable label is a fluorophore. In some embodiments, the first detectable label, the second detectable label, and/or the third detectable label are the same. In some embodiments, the first detectable label, the second detectable label, and/or the third detectable label are different. In some embodiments, the first detectable label, the second detectable label, and/or the third detectable label are detected on the substrate. In some embodiments, the first detectable label, the second detectable label, and/or the third detectable label are compared to generate a spatial fragment DV heat map. In some embodiments, the first detectable label, the second detectable label, and/or the third detectable label are compared to generate a spatial fragment DV number. In some embodiments, the first detectable probe, the second detectable probe, and the third detectable probe are contacted with the substrate sequentially, with disassociation of the previously applied probe as further described herein. In some embodiments, the first detectable probe, the second detectable probe, and the third detectable probe are contacted with the substrate simultaneously.

In some embodiments, detectable probes detect short single-stranded cDNA (e.g., cDNA generated from 18S rRNA), or a complement thereof. In some embodiments, a “short” single-stranded cDNA, or a complement thereof, includes a cDNA about 60 nucleotides or less from the 3′ end of the captured analyte. Thus, for example, a detectable probe designed to detect a short cDNA (e.g., an extended capture probe), or a complement thereof, can be designed to detect a single-stranded cDNA sequence, or complement thereof, between position 1 and position 60 (e.g., short extended capture probe) from the 3′ end of the captured analyte (e.g., 18S rRNA), or complement thereof. In some embodiments, detectable probes detect mid-length extended capture probes (e.g., single stranded cDNA generated from 18S rRNA), or a complement thereof. In some embodiments, a “mid-length” extended capture probe (e.g., single-stranded cDNA) includes cDNA that includes a sequence, or complement thereof, from about 120 nucleotides to about 180 nucleotides from the 3′ end of the captured analyte. Thus, for example, a detectable probe designed to detect a mid-length extended capture probe (e.g., single-stranded cDNA) can be designed to detect a single-stranded cDNA sequence, or complement thereof, between position 120 and position 175 from the 3′ end of the captured analyte (e.g., 18S rRNA). In some embodiments, the one or more second probes detect a mid-length extended capture probe, or complement thereof. In some embodiments, the one or more second detectable probes are positioned 5′ to the location of the first detectable probe. In some embodiments, a “long” extended capture probe (e.g., single-stranded cDNA) includes a sequence, or complement thereof, from about 180 nucleotides to about 220 nucleotides from the 3′ end of the captured analyte. Thus, for example, a detectable probe designed to detect a long extended capture probe (e.g., single-stranded cDNA) can be designed to detect a single-stranded cDNA sequence, or complement thereof, between position 180 and position 220 (or more) from the 3′ end of the captured analyte (e.g., 18S rRNA). In some embodiments, the one or more third detectable probes detect a long extended capture probe, or complement thereof. In some embodiment, the one or more detectable probes are located at a position 5′ to the second detectable probe. For example, an extended capture probe (e.g., single-stranded cDNA), or complement thereof, including a sequence of 100 nucleotides of the captured analyte, or a complement thereof, can be detected by a detectable probe designed to hybridize to the short extended capture probe, but would not be detected by a detectable probe designed to detect a long extended capture probe (e.g., single-stranded cDNA), or a complement thereof. Conversely, an extended capture probe (e.g., a single-stranded cDNA), or a complement thereof, including a sequence of 250 nucleotides of the captured analyte, or a complement thereof, can be detected by a detectable probe designed to hybridize to a short extended capture probe (e.g., single-stranded cDNA), a mid-range extended capture probe, and a long extended capture probe, or complements thereof.

In some embodiments, the first detectable probes are contacted with the biological sample and specifically bind to the extended capture probe (e.g., single-stranded cDNA), or complement thereof, and are detected (e.g., fluorescence is detected from the detectable label) and the image is recorded. In some embodiments, the first detectable probe can be disassociated (e.g., removed) and the process is repeated for a second, a third, or more detectable probes. Thus, for example, the recorded images from each of the detectable probes can be compared to generate a spatial fragment (DV) heat map. In some embodiments, the spatial fragment DV heat map can represent the level of nucleic acid degradation present in the biological sample. In some embodiments, the spatial fragment DV heat map can be represented as one or more spatial fragment DV numbers (e.g., 1 to 100) for the individual detectable probes. For example, a biological sample can have one or more spatial fragment DV numbers that correspond to the location where the one or more detectable probes hybridized to the extended capture probe (e.g., single-stranded cDNA), or complement thereof. For example, a biological sample can have one or more spatial fragment DV numbers that correspond with the contacted first, second, and third detectable probes designed to detect short, mid-range, and long extended capture probes, or complements thereof, respectively.

In some embodiments, a spatial fragment DV number for a long extended capture probe (e.g., single-stranded cDNA), or a complement thereof, is indicative of RNA of sufficient integrity (e.g., lack of degradation) for other downstream analyses, such as spatial transcriptomics, can be from about 60 to about 100, about 65 to about 95, about 70 to about 90, about 75 to about 85, or about 70. In some embodiments a spatial fragment DV number for a long single-stranded cDNA (e.g., extended capture probe), or complement thereof, indicative of RNA integrity sufficient for other downstream analyses, such as spatial transcriptomics, can be about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100. In some embodiments a biological sample can have a low (e.g., less than 7) RNA integrity number or RIN score. High quality RNA is defined as full-length (or close to full-length) transcripts, whereas low quality RNA is defined as fragmented transcripts. RIN values range from 1 to 10, with higher numbers indicating higher quality (e.g., less degraded, less fragmented) RNA samples. In some embodiments, a biological sample can have a spatial fragment DV number for a long single-stranded cDNA less than 60 and a RIN scope of less than 7, where both assays indicate that a biological sample contains degraded RNA of insufficient integrity for other downstream applications. In some embodiments a biological sample can have a low (e.g., less than 7) RIN score and a spatial fragment DV number for a long single-stranded cDNA, or complement thereof, of 60 or above. Thus, for example, an RNA integrity assay, such as a spatial fragment DV assay, can identify biological samples (e.g., fixed biological samples) that may contain RNA of sufficient integrity for further downstream analyses not identified by a RIN score.

Provided herein are arrays including a plurality of capture probes, where a capture probe of the plurality of capture probes includes a capture domain that is capable of specifically binding to a sequence present in an 18S ribosomal RNA. In some embodiments, the capture probe further includes a spatial barcode (e.g., any of the spatial barcodes described herein). In some embodiments, the capture probe further includes one or more functional domains, a cleavage domain, a unique molecular identifier, and combinations thereof.

Also provided herein are kits including an array (e.g., any of the arrays described herein), a first detectable probe, where the first detectable probe includes a sequence that corresponds to a first sequence present in a 3′ region of the rRNA and a first detectable label, and a second detectable probe, where the second detectable probe includes a sequence that corresponds to a second sequence in the rRNA that is positioned 5′ relative to the first sequence in the rRNA, and a second detectable label. In some embodiments of the kits described herein, one or both of the first detectable label and the second detectable label is a fluorophore. In some embodiments of the kits described herein, the first detectable label and the second detectable label are different. In some embodiments the first detectable label and the third detectable label are the same.

In some embodiments of any of the kits described herein, a third detectable probe is included, where the third detectable probe includes a sequence that corresponds to a third sequence in the rRNA that is positioned 5′ relative to the second sequence in the rRNA and a third detectable label. In some embodiments, third detectable label is a fluorophore. In some embodiments of the kits described herein, the first detectable label, the second detectable label, and the third detectable label are different. In some embodiments of the kits described herein, the first detectable label, the second detectable label, and the third detectable label are the same. In some embodiments of the kits described herein, one or more permeabilization reagents is included. In some embodiments of the kits described herein, the one or more permeabilization reagents comprises TE buffer. In some embodiments of the kits described herein a protease (e.g., pepsin, Proteinase K, any of the other proteases described herein) is included. In some embodiments of the kits described herein, a nuclease (e.g., RNase, RNase H). In some embodiments of the kits described herein a reverse transcriptase is included.

### EXAMPLES

### Example 1: Spatial Fragment Distribution Value Probe Hybridization Efficiency

Probe Design

Primer3 Software (version 4.1.0) was used for designing the capture, hybridization, surface and control probes. The positions of the sequences were selected to be compatible with both mouse (NR 003278.3) and human (NR 003286.4) 18S rRNA. Hybridization probes (DV probes) were designed with complementary sequences to the synthesized 18S cDNA. The capture probe was designed to contain complementary sequence to the 3′ end of human 18S rRNA and functioned as a template to reverse transcribe the 18S rRNA.

Printing Probes

Manufacturer's instructions were followed to place the probes on CodeLink Activated slides. The probes included 5′ amino C6 modification. Two mixtures each containing 0.06% Sarkosyl and 20 μM of control probe or capture probe were prepared according to Surmodics instructions. All probes were evaluated using Oligo Calc: Oligonucleotide Properties Calculator for detecting potential self-annealing positions or hairpin formation. Moreover, potential secondary structures of the probes along with their associated free energies were estimated through mFold web server, and probe designs demonstrating secondary structures with energies greater than −6 kcal/mol were excluded. To ensure specific binding and predict any unexpected hybridization patterns, the NCBI BLAST database was also queried.

Probes and primers used in the spatial fragment distribution value (sDV) assay were designed (Table 1) and validated under various conditions.

TABLE 1

| | Position into the 3′ | | |
|---|---|---|---|
| Probe | end of 18s rRNA | 5′ end modification | SEQ ID NO: |
| Control Probe | — | Amino-C6 | 1 |
| Surface Probe | — | 5Cy5 | 2 |
| DV59 Probe | 59 | 5Cy3 | 3 |
| DV150 Probe | 150 | 5Cy3 | 4 |
| DV195 Probe | 195 | 5Cy3 | 5 |
| Capture Probe | 19 | Amino-C6 | 6 |

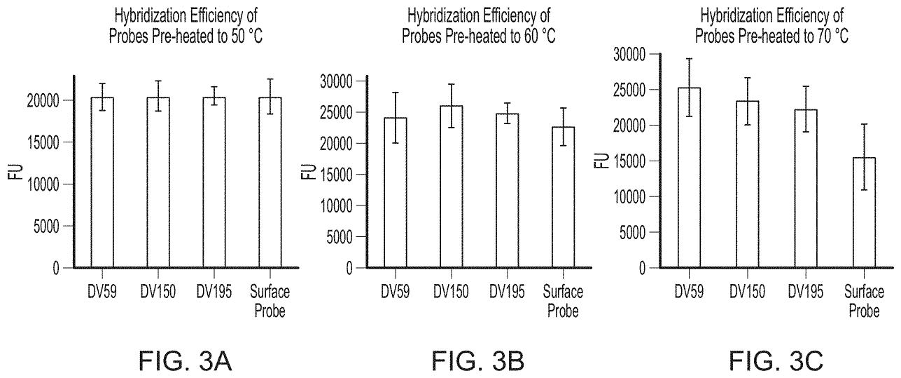

The surface probe (SEQ ID NO: 2) functions as a control and is complementary to the capture probe (SEQ ID NO: 6). The control probe (SEQ ID NO: 1) includes complementary sequences to the DV59 probe (SEQ ID NO: 3), the DV150 probe (SEQ ID NO: 4), and the DV195 probe (SEQ ID NO: 5) (“DV probes”). The DV59, DV150, and DV195 probes are Cy3 labeled at their 5′ ends to allow for detection when hybridized to the control probe. The positions listed in Table 1 refer to the positions of the sequences relative to the human 18S rRNA molecule (NR 003286.4) with a total length of 1869 bases. To validate detection of the DV probes, the control probe was printed under identical conditions in 4 spots in each well of a slide and fluorescence was detected (data not shown). FIGS. 3A-C show results of hybridization efficiency of the detection probes (DV probes) and the surface probe at different temperatures (50° C., 60° C., and 70° C.) as measured in fluorescence units (FU). DV probes were pre-heated to 50° C. (surface probes were pre-heated to 70° C.) demonstrated similar hybridization patterns with relatively low standard deviations. In contrast, pre-heating the DV probes to 60° C. or higher (including 70° C.), resulted in higher fluorescence signals for the DV probes, but with less uniform patterns and higher standard deviations.

### Example 2: RNA Integrity in Bulk Samples

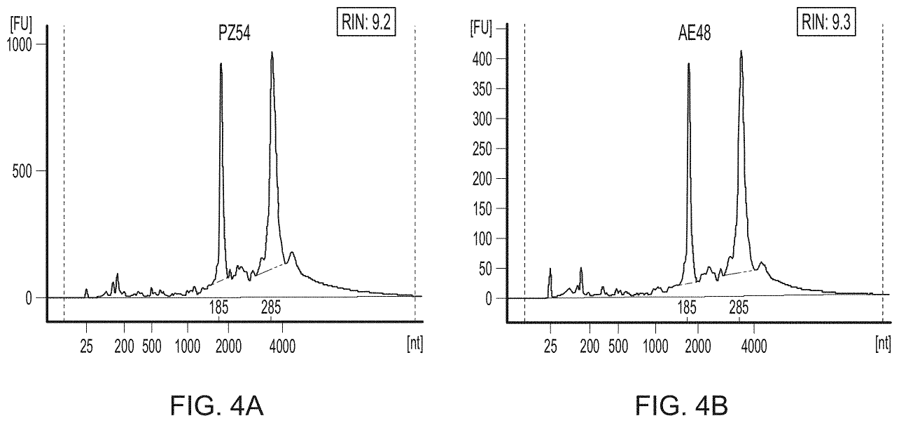

RNA integrity assays were performed on fresh frozen (control) and FFPE samples from the same individual(s). FIGS. 4A & 4B show RIN values of 9.2 and 9.3 from control fresh frozen samples, respectively, from two different individuals demonstrating a high RIN value typically observed in fresh frozen biological samples. The gel electropherograms in FIGS. 4A-B show 18S and 28S ribosomal RNA peaks are indicative of intact RNA content of the control fresh frozen samples.

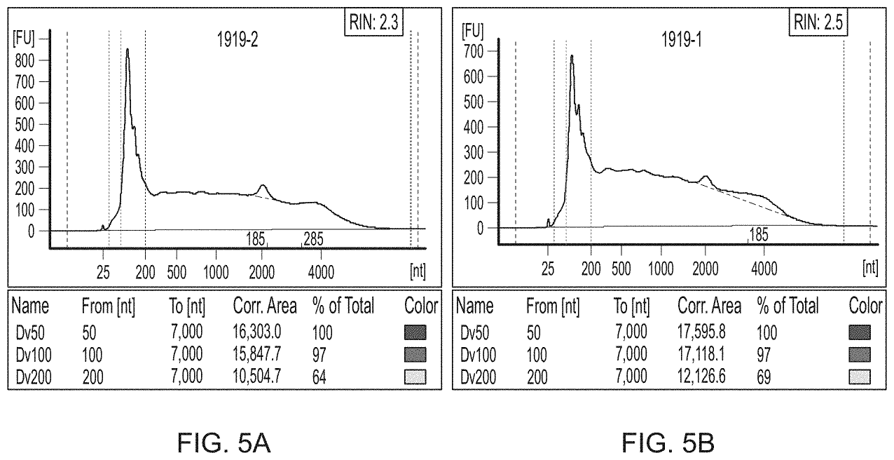

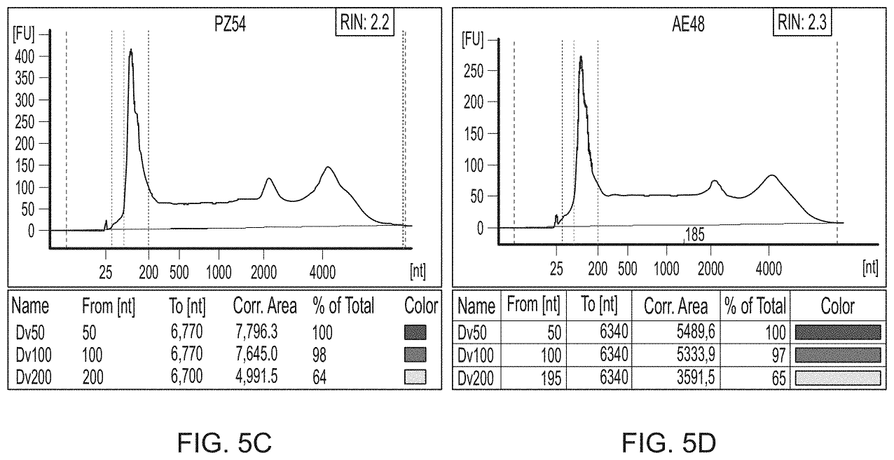

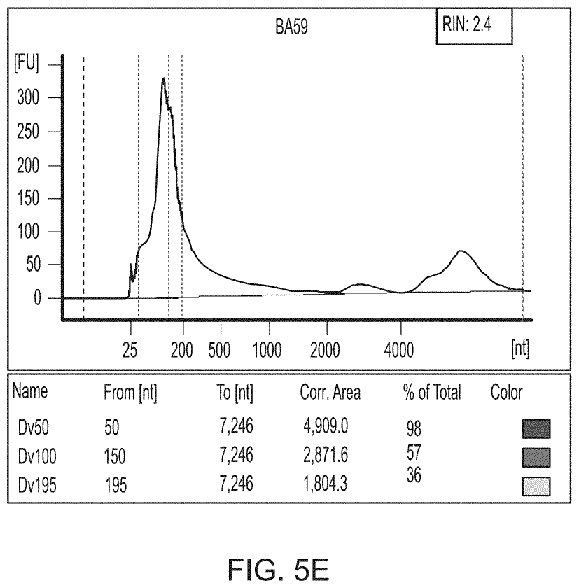

FIGS. 5A-E show both RIN values and DV values in FFPE samples from different individuals, including FFPE samples from the individuals who provided fresh frozen samples that resulted in the data shown in FIG. 4A-B (FIG. 5C and FIG. 5D, respectively). The RIN values in the FFPE biological samples ranged from 2.2 to 2.5, indicating RNA that may be too degraded for downstream applications. The spatial fragment DV numbers varied with the individual DV probe lengths, they mainly reported RNA that was of sufficient integrity for downstream applications. For example, for FIGS. 5A-D, the DV50 spatial fragment DV number was measured at 100% of total in each of the four FFPE biological samples, the DV100 values ranged from 97-98% and the DV200 values ranged from 64-69%. As such, while the RIN numbers for FIGS. 5A-D suggest that the RNA may be degraded, the spatial fragment DV values suggest otherwise. However, FIG. 5E reports a RIN of 2.4, a DV50 of 98%, DV150 of 57% and DV195 of 36%, pointing to a sample where the RNA may not be of sufficient integrity for downstream applications. The electropherograms shown in FIGS. 5A-E are characterized by different peaks compared to their paired fresh frozen samples (FIG. 4A-B). Both 18S and 28S ribosomal RNA peaks were found to be more challenging to observe in the FFPE electropherograms as they exhibited reduced peak height and area. However, the DV195 values measured in four out of five FFPE samples were 64% or higher.

### Example 3: Spatial Distribution Values in FFPE Biological Samples

RNA Extraction

Total RNA was extracted from FFPE specimens (RNeasy FFPE Kit (QIAGEN)) according to the manufacturer's instructions. An additional 10 min incubation step at room temperature prior to the final dilution of RNA was performed. The samples were eluted with 14 μl RNase-free water and RNA quality determination was performed on an Agilent 2100 Bioanalyzer system (Agilent Technologies) with an Agilent RNA 6000 Pico Kit.

Human reference total RNA was purified and prepared according to the following method: reference RNA was centrifuged at 12,000 g for 15 min at 4° C. Supernatant was removed and the pellet was washed with 70% ETOH solution. The reference RNA was centrifuged again. The supernatant was removed and the tube was left to air-dry at room temperature for 30 min. The pellet was resuspended in 200 μl RNase free water, vortexed and spun down.

Tissue Sectioning

Specimens were placed on ice for 10 min prior to sectioning. The specimens were sectioned at 12 μm thickness with a microtome. Sections were floated in a DNase/RNase free water bath at 45° C., placed on a slide, and left to dry in an oven at 37° C. for 2 hours.

Deparaffinization and Crosslink Reversal

To deparaffinize the tissues the slides were incubated two times in Xylene for 5 min at room temperature, followed by incubation in 99.5% EtOH for 2 min at room temperature, and again in 99.5% EtOH for 2 min. Next, the slides incubated in two times in 96% EtOH for 2 min. Crosslink reversal was performed by adding 75 μl of TE buffer (pre-heated to 70° C.) to each well and incubated at 70° C. for 60 minutes. After incubation, the wells were washed with 100 μl of 0.1×SSC.

H&E Staining and Bright Field Imaging

Propan-2-ol was added to the tissue prior to H&E staining and left at room temperature until evaporation. Mayer's Hematoxylin (S3309, Agilent) was added to the tissue incubated for 7 min. Next, the tissues were washed with nuclease free water and incubated in Bluing buffer for 2 min and washed again in nuclease free water. The tissues were stained with Eosin diluted in Tris acetic acid of pH 6.0 for 60 seconds, washed in water, and incubated to dry for 5 min at 37° C. The slides were mounted in 85% glycerol and covered with a coverslip. Metafer Slide Scanning system and Vslide software (MetaSystems) were used to generate the bright field images.

Permeabilization

Permeabilization was performed in a hybridization cassette (AHC1X16, ArrayIT Corporation) with 0.2 U/μl Collagenase Type 1, HBSS buffer, and 0.2 mg/μl BSA at 37° for 20 min. After incubation the tissues were washed in 0.1×SSC buffer and a mixture of 0.1% pepsin dissolved in 0.1M HCl at 37° for 10 min. The mixture was then washed out using 0.1×SSC buffer. Reference total RNA sample was added to a well on the slide and left to dry.

Reverse Transcription

Reverse transcription was performed with 70 μl of the reaction mixture at 42° C. overnight. The reverse transcription reaction mixture contained the following: 1× First strand buffer, 1 M Betaine, 6 mM MgCl2s, 0.2 mg/ml BSA, 50 ng/μl Actinomycin D, 5 mM DTT, 10% DMSO, 1 mM dNTPs, 2 U/μl RNaseOUT Recombinant Ribonuclease Inhibitor, 20 U/μl SuperScript® III Reverse Transcriptase. The next day the reaction mixture was removed and the tissue was washed using 0.1×SSC buffer.

Tissue and Ribosomal RNA Removal

Tissues were incubated with β-Mercaptoethanol in a buffer (ratio 3:100) for 60 min at 56° C. with continuous shaking (300 rpm). Then the tissues were incubated with Proteinase K in Proteinase K digest buffer (PKD buffer) (ratio of 1:3) for 2 hours at 56° C. with gentle shaking. The hybridization cassette was removed and the array was washed with continuous shaking at 300 rpm with 2×SSC in 0.1% SDS at 50° for 10 min, followed by 0.2×SSC at room temperature for 1 min, and with 0.1×SSC at room temperature for 1 min. The arrays were spun dried and returned to the hybridization cassette. The rRNA removal mixture (1× First strand buffer, 0.4 mg/ml BSA and X U/ml RNase H) was added to the array under intervals of gentle shaking at 300 rpm, for 60 min at 37° C., followed by a wash in 0.1×SSC buffer. Finally, a 60% DMSO treatment for 5 min at room temperature, followed by 3 additional wash steps with 0.1×SSC was performed.

Hybridization of Detection Probes

To validate the DV probes hybridization efficiency, hybridization mixtures (1 mM EDTA, 50 mM NaCl, 10 mM Tris-HCl and 0.5 μM fluorescently labelled probes) were prepared and pre-heated to either 50° C., 60° C., or 70° C. The pre-heated mixture was added to each well in the hybridization cassette and incubated for 10 min at room temperature. Following incubation the mixture was removed and the array was washed in 2×SSC and 0.1% SDS at 50° C. for 10 min with continuous shaking at 300 rpm, followed by a wash with 0.2×SSC for 1 min, and a wash with 0.1×SSC for 1 min at room temperature. The slides were spun dried and stored away from light. The spatial fragment DV assays were performed at 50° C.

Imaging and Dehybridization of Detection Probes

A DNA microarray scanner (InnoScan 910, Innopsys) was used with gain of 20 and then set to 70 for 532 nm wavelength excitation to scan and image the slides for the spatial fragment DV assays. Gains of 1, 20 and 70 were used for excitation of at 635 nm. A manual alignment of the tif files was performed (Adobe Photoshop CC 2020) for use in the RIN Script to generate spatial fragment DV heat maps. A DNA microarray scanner (InnoScan 910, Innopsys) was used with gain of 0.5 for 532 nm wavelength excitation and gain 1 for 635 nm excitation to scan and image the control probe (hybridization efficiency experiment) and fluorescence signal analysis was performed with Mapix (Innopsys) software.

Between hybridization rounds of the DV probes, dehybridization was performed by incubating the array for 5 min with 60% DMSO at room temperature, followed by three washes in 0.1×SSC buffer.

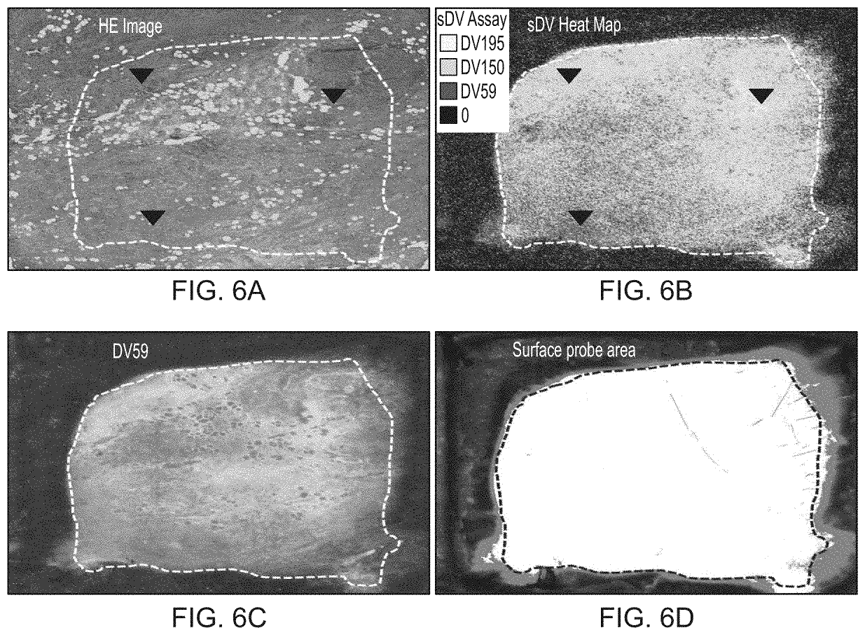

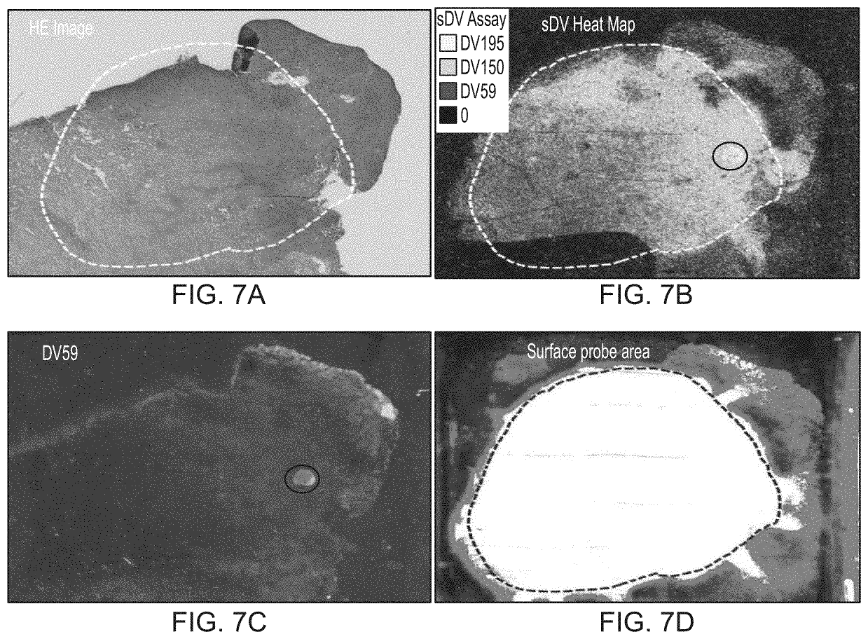

FIGS. 6A-D and FIGS. 7A-D show spatial (DV) heat maps from two different individuals. FIGS. 6A-D shows various images from individual “AE48” FFPE samples (see also, FIG. 4B and FIG. 5D), including image “A” showing H&E staining, image “B” showing a spatial (DV) heat map with three differentially lengthened DV probes (DV59, 150 and 195), image “C” showing the DV59 probe alone, and image “D” showing a control where the surface probe is hybridized to the capture probe (no DV probes). Similarly, FIGS. 7A-D shows various images from individual “BA59” FFPE samples (see also, FIG. 5E) including image “A” showing H&E staining, image “B” shows a spatial (DV) heat map with all three DV probes, image “C” showing the DV195 probe alone (non-hashed circle indicates an artifact), and image “D” showing a control where the surface probe is hybridized to the capture probe (no DV probes).

In both FIGS. 6A-B and 7A-B the area outlined by the dotted line in the bright field images and the spatial fragment DV heat maps correspond to a capture area (e.g., where the capture probes were printed). As expected, fluorescent signals were detected inside the dotted line for all except the control “D”. The capture area was defined by detecting fluorescence of the surface probe and scanning for Cy5 fluorescent signal (FIG. 6D and FIG. 7D).

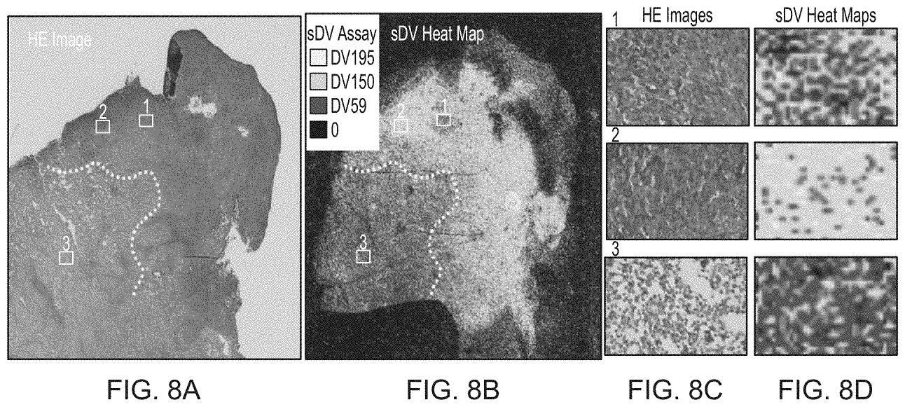

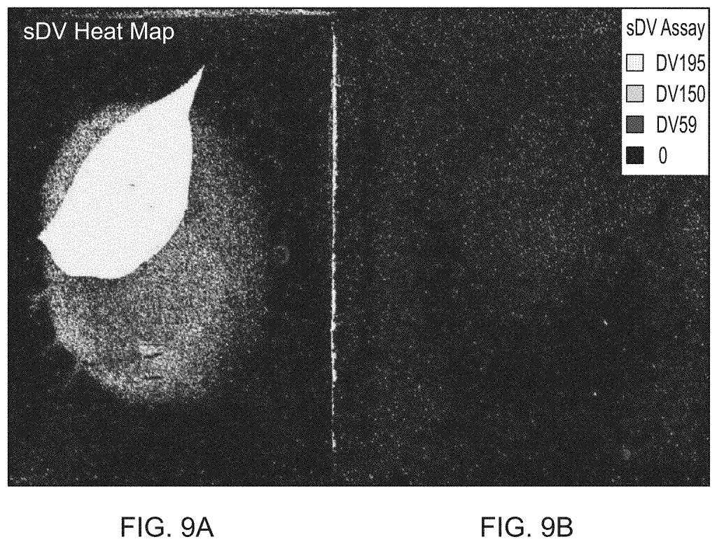

FIGS. 8A-D show images from FFPE biological samples prepared from individual AE48 including image “A” showing H&E staining, image “B” showing a spatial (DV) heat map with all three spatial fragment DV probes, image “C” showing close-up H&E staining from three specific regions of the FFPE biological sample (denoted 1, 2, and 3 on FIGS. 8A and 8B), and image “D” showing close-up imaging of the spatial (DV) heat map from regions 1, 2, and 3. The dotted line shown in FIGS. 8A and 8B divides the tissue into two regions showing varied degrees of RNA degradation, which is more clearly observed in close-up images FIGS. 8C and 8D. For example, the spatial fragment DV heat map in FIG. 8D shows primarily DV59 (blue) detection and less of either the DV150 (green) or DV195 probe (yellow), indicative of shorter cDNA length and thus more RNA degradation. FIGS. 9A and 9B show positive and negative controls, respectively, where FIG. 9A contained a human reference RNA sample and FIG. 9B contained no biological sample.

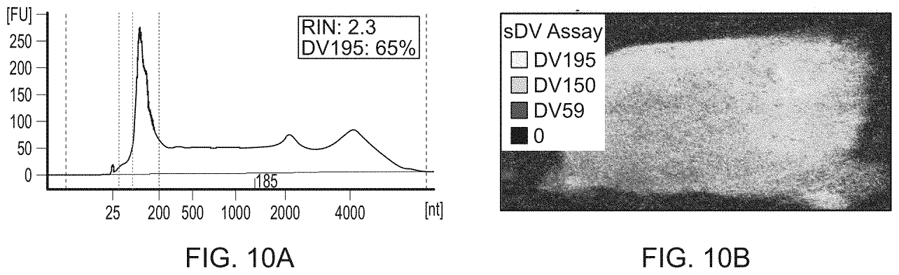

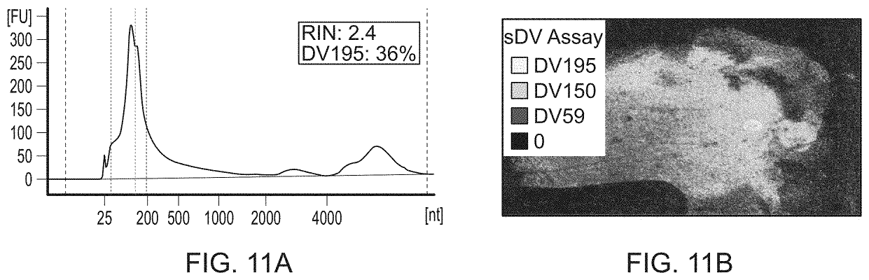

FIGS. 10A-B and FIG. 11A-B show side-by-side comparison of RIN analysis and spatial fragment DV heat maps from two different individuals. The RIN values for both samples are indicative of highly degraded RNA (RIN values 2.3 and 2.4, respectively), whereas the DV195 value in the two samples were 65% and 36%, respectively. Using the DV195 value as a measurement of longer (e.g., increased integrity) RNA suggests that samples with lower RIN values may actually be analyzable either in bulk or spatially.

Sequence Listing
Control Probe
SEQ ID NO: 1
UUUUACGACTTTTACTTCCTCTAGGCGTATGCGGATTGGGCTCCTCACTA
AACCATCCAAGCGTATGCGGATTGGGCTAAAGGGCAGGGACTTAAT
Surface Probe
SEQ ID NO: 2
CAAGGTTTCCGTAGGTGA
DV59 Probe
SEQ ID NO: 3
CTAGAGGAAGTAAAAGTCGT
DV150 Probe
SEQ ID NO: 4
TTGGATGGTTTAGTGAGG
DV195 Probe
SEQ ID NO: 5
ATTAAGTCCCTGCCCTTT
Capture Probe
SEQ ID NO: 6
UUUUUTCACCTACGGAAACCTTG

### EMBODIMENTS

Embodiment 1 is a method of determining the presence of RNA of sufficient integrity suitable for downstream applications in a fixed biological sample, the method comprising: (a) generating a spatial fragment distribution value (DV) number of the fixed biological sample; (b) generating an RNA integrity number (RIN) score of the FFPE biological sample; and (c) using the generated spatial fragment distribution value (DV) number of step (a), and the RIN score of the fixed biological sample of step (b), to determine the presence of RNA of sufficient integrity suitable for downstream applications in the fixed biological sample.

Embodiment 2 is the method of embodiment 1, wherein determine the spatial fragment DV number comprises: (a) contacting the fixed biological sample with a substrate comprising a plurality of capture probes, wherein a capture probe of the plurality of capture probes comprises a capture domain that is capable of binding specifically to a ribosomal RNA (rRNA) from the fixed biological sample; (b) de-crosslinking one or more crosslinks in the fixed biological sample; (c) permeabilizing the fixed biological sample under conditions sufficient to allow the rRNA to bind specifically to the capture domain; (d) extending an end of the capture probe using the rRNA specifically bound to the capture domain as a template, thereby generating an extended capture probe; (e) contacting a first detectable probe to the extended capture probe, wherein the first detectable probe comprises (i) a sequence that corresponds to a first sequence present in a 3′ region of the rRNA, and (ii) a first detectable label; and (f) detecting a location of the first detectable label on the substrate, thereby determining a spatial fragment DV number of the first detectable label.

Embodiment 3 is the method of embodiment 2, wherein extending the end of the capture probe comprises extending a 3′ end of the capture probe.

Embodiment 4 is the method of embodiment 2 or 3, wherein extending the 3′ end of the capture probe comprises generating a single-stranded cDNA.

Embodiment 5 is the method of any one of embodiments 2-4, wherein the method further comprises, before step (c), a step of staining and imaging the fixed biological sample.

Embodiment 6 is the method of embodiment 5, wherein the fixed biological sample is stained with hematoxylin and eosin.

Embodiment 7 is the method of any one of embodiments 2-6, wherein the rRNA is an 18S rRNA.

Embodiment 8 is the method of any of embodiments 2-6, wherein the rRNA is a 28S rRNA.

Embodiment 9 is the method of any one of embodiments 2-8, wherein the de-crosslinking step comprises heating the fixed biological sample.

Embodiment 10 is the method of any one of embodiments 2-9, wherein the de-crosslinking step comprises the performance of a chemical reaction.

Embodiment 11 is the method of any one of embodiments 2-10, wherein the de-crosslinking step comprises the use of an enzyme.

Embodiment 12 is the method of any one of embodiments 2-11, wherein the de-crosslinking step comprises the use of TE buffer.

Embodiment 13 is the method of embodiment 12, wherein the TE buffer has a temperature of about 65° C. to about 75° C., and is contacted with the fixed biological sample for about 30 minutes to about 90 minutes.

Embodiment 14 is the method of any one of embodiments 2-13, wherein the step of extending the end of the capture probe is performed in the presence of actinomycin D.

Embodiment 15 is the method of any one of embodiment 2-13, wherein the method further comprises treating the fixed biological sample with an RNase after step (d).

Embodiment 16 is the method of embodiment 15, wherein the RNase is RNase H.

Embodiment 17 is the method of any one of embodiments 2-16, wherein the step of permeabilizing the fixed biological sample includes the use of a protease.

Embodiment 18 is the method of embodiment 17, wherein the protease is pepsin or proteinase K.

Embodiment 19 is the method of any one embodiments 2-18, wherein the fixed biological sample is removed after the extending in step (d).

Embodiment 20 is the method of embodiments 2-19, further comprising: contacting a second detectable probe to the extended capture probe, wherein the second detectable probe comprises (i) a sequence that corresponds to a second sequence in the rRNA that is positioned 5′ relative to the first sequence in the rRNA, and (ii) a second detectable label; and detecting a location of the second detectable label on the substrate, thereby determining a spatial fragment DV number of the second detectable label.

Embodiment 21 is the method of embodiments 2-20, wherein the method further comprises contacting a third detectable probe to the extended capture probe, wherein the third detectable probe comprises (i) a sequence that corresponds to a third sequence in the rRNA that is positioned 5′ relative to the second sequence in the rRNA, and (ii) a third detectable label; and detecting a location of the third detectable label on the substrate, thereby determining a spatial fragment DV number of the third detectable label.

Embodiment 22 is the method of any one of embodiments 2-21, wherein the first detectable label, the second detectable label, and the third detectable label is a fluorophore.

Embodiment 23 is the method of any one of embodiments 2-22, wherein the first detectable label, the second detectable label, and the third detectable label are different.

Embodiment 24 is the method of any one of embodiments 2-22, wherein the first detectable label, the second detectable label, and the third detectable label are the same.

Embodiment 25 is the method of any one of embodiments 2-24, wherein the method further comprises, a step of disassociating and removing the first detectable probe from the extended capture probe prior to contacting the substrate with the second detectable probe.

Embodiment 26 is the method of any one of embodiments 2-25, wherein the method further comprises, a step of disassociating and removing the second detectable probe from the extended capture probe prior to contacting the substrate with the third detectable probe.

Embodiment 27 is the method of any one of embodiments 1-26, wherein the first detectable probe detects a short extended capture probe.

Embodiment 28 is the method of embodiment 27, wherein the short extended capture probe comprises an extended capture probe of about 60 nucleotides or less from the 3′ end of the captured analyte.

Embodiment 29 is the method of any one of embodiments 1-26, wherein the second detectable probe detects a mid-length extended capture probe.

Embodiment 30 is the method of embodiment 29, wherein the mid-length extended capture probe comprises an extended capture probe from at least about 120 nucleotides to about 180 nucleotides from the 3′ end of the captured analyte.

Embodiment 31 is the method of any one of embodiments 1-26, wherein the third detectable probe detects a long extended capture probe.

Embodiment 32 the method of embodiment 31, wherein the long extended capture probe comprises an extended capture probe from at least about 180 nucleotides to about 220 nucleotides from the 3′ end of the captured analyte.

Embodiment 33 is the method of any one embodiments 1-32, wherein the spatial fragment DV number of the FFPE biological sample comprises a number between 1 and 100.

Embodiment 34 is the method of any one of embodiments 1-33, wherein the spatial fragment DV number of the long extended capture probe comprises 60 or greater.

Embodiment 35 is the method of embodiment 34, wherein the spatial fragment DV number of 60 or greater of the long extended capture probe or greater is indicative of RNA of sufficient integrity suitable for downstream applications.

Embodiment 36 is the method of any of embodiments 1-35, wherein generating the RIN score for the fixed biological sample comprises a score between 1 and 10.

Embodiment 37 is the method of 36, wherein the RIN score of 7 or greater is indicative of RNA of sufficient integrity suitable for downstream applications.

Embodiment 38 is the method of any one of embodiments 1-37, wherein the fixed biological sample comprises the spatial fragment DV number of the long extended capture probe of less than 60 and the RIN score of less than 7.

Embodiment 39 is the method of any one of embodiments 1-37, wherein the fixed biological sample comprises the spatial fragment DV number of the long extended capture probe of 60 or greater and the RIN score of less than 7.

Embodiment 40 is the method of any one embodiments 1-39, wherein a downstream application comprises spatial transcriptomics.

Embodiment 41 is the method of any one of embodiments 1-40, wherein the fixed sample is a formalin-fixed paraffin-embedded sample biological sample, a PFA fixed biological sample, or an acetone fixed biological sample.

Embodiment 42 is the method of any one of embodiments 1-41, wherein the fixed biological sample is an FFPE tissue section, a PFA tissue section, or an acetone fixed tissue section.

Embodiment 43 is the method of any one of embodiments 1-42, wherein the fixed biological sample is a tumor sample.

Embodiment 44 is a method for generating a spatial fragment distribution value (DV) heat map of a fixed biological sample, the method comprising: (a) contacting the fixed biological sample with a substrate comprising a plurality of capture probes, wherein a capture probe of the plurality of capture probes comprises a capture domain that is capable of binding specifically to a ribosomal RNA (rRNA) from the fixed biological sample; (b) de-crosslinking one or more crosslinks in the fixed biological sample; (c) permeabilizing the fixed biological sample under conditions sufficient to allow the rRNA to bind specifically to the capture domain; (d) extending an end of the capture probe using the rRNA specifically bound to the capture domain as a template, thereby generating an extended capture probe; (e) contacting a first detectable probe to the extended capture probe, wherein the first detectable probe comprises (i) a sequence that corresponds to a first sequence present in a 3′ region of the rRNA, and (ii) a first detectable label; (f) detecting a location of the first detectable label on the substrate; (g) contacting a second detectable probe to the extended capture probe, wherein the second detectable probe comprises (i) a sequence that corresponds to a second sequence in the rRNA that is positioned 5′ relative to the first sequence in the rRNA, and (ii) a second detectable label; (h) detecting a location of the second detectable label on the substrate; and (i) comparing the location of the first detectable label to the location of the second detectable label on the substrate, thereby generating the spatial fragment DV heat map of the fixed biological sample.

Embodiment 45 is the method of embodiment 44, wherein extending the end of the capture probe comprises extending a 3′ end of the capture probe.

Embodiment 46 is the method of embodiment 44 or 45, wherein extending the 3′ end of the capture probe comprises generating a single-stranded cDNA.

Embodiment 47 is the method of any one of embodiments 44-46, wherein the method further comprises, before step (c), a step of staining and imaging the fixed biological sample.

Embodiment 48 is the method of embodiment 47, wherein the fixed biological sample is stained with hematoxylin and eosin.

Embodiment 49 is the method of any one of embodiments 44-48, wherein the rRNA is an 18S rRNA or a 28S rRNA.

Embodiment 50 is the method of any one of embodiments 44-49, wherein the de-crosslinking step comprises heating the fixed biological sample.

Embodiment 51 is the method of any one of embodiments 44-50, wherein the de-crosslinking step comprises the performance of a chemical reaction.

Embodiment 52 is the method of any one of embodiments 44-51, wherein the de-crosslinking step comprises the use of an enzyme.

Embodiment 53 is the method of any one of embodiments 44-52, wherein the de-crosslinking step comprises the use of TE buffer.

Embodiment 54 is the method of embodiment 53, wherein the TE buffer has a pH of about 7.5 to about 8.5.

Embodiment 55 is the method of embodiment 54, wherein the TE buffer has a temperature of about 65° C. to about 75° C., and is contacted with the fixed biological sample for about 30 minutes to about 90 minutes.

Embodiment 56 is the method of any one of embodiments 44-55, wherein the step of extending the end of the capture probe is performed in the presence of actinomycin D.

Embodiment 57 is the method of any one of embodiment 44-56, wherein the method further comprises treating the fixed biological sample with an RNase after step (d).

Embodiment 58 is the method of embodiment 57, wherein the RNase is RNase H.

Embodiment 59 is the method of any one of embodiments 44-58, wherein the step of permeabilizing the FFPE biological sample includes the use of a protease.

Embodiment 60 is the method of embodiment 59, wherein the protease is pepsin or proteinase K.

Embodiment 61 is the method of any one embodiments 44-60, wherein the fixed biological sample is removed after the extending in step (d).

Embodiment 62 is the method of any one of embodiments 44-61, wherein one or both of the first detectable label and the second detectable label is a fluorophore.

Embodiment 63 is the method of embodiment 62, wherein step (f) and/or step (h) comprises detecting fluorescence of the first and/or second detectable label.

Embodiment 64 is the method of any one of embodiments 44-63, wherein the first detectable label and the second detectable label are different.

Embodiment 65 is the method of any one of embodiments 44-63, wherein the first detectable label and the second detectable label are the same.

Embodiment 66 is the method of any one of embodiments 44-65, wherein the method further comprises, between steps (g) and (h), a step of disassociating and removing the first detectable probe from the extended capture probe.

Embodiment 67 is the method of any one of embodiments 44-66, wherein the method further comprises, between steps (h) and (i), a step of disassociating and removing the second detectable probe from the extended capture probe.

Embodiment 68 is the method of embodiment 67, wherein the method further comprises contacting a third detectable probe to the extended capture probe, wherein the third detectable probe comprises (i) a sequence that corresponds to a third sequence in the rRNA that is positioned 5′ relative to the second sequence in the rRNA, and (ii) a third detectable label.

Embodiment 69 is the method of embodiment 68, wherein the method further comprises detecting a location of the third detectable label on the substrate.

Embodiment 70 is the method of embodiment 68 or 69, wherein the first detectable label, the second detectable label, and the third detectable label are different.

Embodiment 71 is the method of embodiment 69 or 70, wherein the first detectable label, the second detectable label, and the third detectable label are the same.

Embodiment 72 is the method of any one of embodiments 68-71, wherein step (i) further comprises comparing the location of the first detectable label on the substrate, the second detectable label on the substrate, and the location of the third detectable label on the substrate, thereby generating the spatial fragment DV heat map of the FFPE biological sample.

Embodiment 73 is the method of any one of embodiments 44-72, wherein the fixed biological sample is a formalin-fixed paraffin-embedded biological sample, a PFA fixed biological sample, or an acetone fixed biological sample.

Embodiment 74 is the method of any one of embodiments 44-73, wherein the fixed biological sample is an FFPE tissue section, a PFA tissue section, or an acetone fixed tissue section.

Embodiment 75 is the method of any one of embodiments 44-74, wherein the fixed biological sample is a tumor sample.

Embodiment 76 is the method of any one of embodiments 44-75, wherein the spatial fragment DV heat map identifies a region of interest in the fixed biological sample.

Embodiment 77 is the method of any one of embodiments 44-76, wherein the method further comprises determining a spatial fragment DV number for the fixed biological sample.

Embodiment 78 is the method of embodiment 77, wherein the spatial fragment DV number is an indication of RNA degradation in the fixed biological sample.

Embodiment 79 is the method of any one of embodiments 44-78, further comprises sequencing the extended capture probe, wherein the extended capture probe comprises a spatial barcode.

Embodiment 80 is an array comprising a plurality of capture probes, wherein a capture probe of the plurality of capture probes comprises a capture domain that is capable of specifically binding to a sequence present in an 18S ribosomal RNA.

Embodiment 81 is the array of embodiment 80, wherein the capture probe further comprises a spatial barcode.

Embodiment 82 is the array of embodiment 81, wherein the capture probe further comprises one or more functional domains, a cleavage domain, a unique molecular identifier, and combinations thereof.

Embodiment 83 a kit comprising: an array of any one of embodiments 80-82; a first detectable probe, wherein the first detectable probe comprises (i) a sequence that corresponds to a first sequence present in a 3′ region of the rRNA, and (ii) a first detectable label; a second detectable probe, wherein the second detectable probe comprises (i) a sequence that corresponds to a second sequence in the rRNA that is positioned 5′ relative to the first sequence in the rRNA, and (ii) a second detectable label.

Embodiment 84 is the kit of embodiment 83, wherein one or both of the first detectable label and the second detectable label is a fluorophore.

Embodiment 85 is the kit of embodiment 83 or 84, wherein the first detectable label and the second detectable label are different.

Embodiment 86 is the kit of embodiment 83 or 84, wherein the first detectable label and the third detectable label are the same.

Embodiment 87 is a kit of any one of embodiments 83-86, wherein the kit further comprises a third detectable probe, wherein the third detectable probe comprises (i) a sequence that corresponds to a third sequence in the rRNA that is positioned 5′ relative to the second sequence in the rRNA, and (ii) a third detectable label.

Embodiment 88 is the kit of embodiment 87, wherein the third detectable label is a fluorophore.

Embodiment 89 is the kit of any one of embodiments 83-85 or 87-88, wherein the first detectable label, the second detectable label, and the third detectable label are different.

Embodiment 90 is the kit of any one of embodiments 83-84 or 86-88, wherein the first detectable label, the second detectable label, and the third detectable label are the same. Embodiment 91 is the kit of any one of embodiments 83-90, further comprising one or more permeabilization reagents.

Embodiment 92 is the kit of embodiment 91, wherein the one or more permeabilization reagents comprises TE buffer.

Embodiment 93 is the kit of any one of embodiments 83-92, further comprising a protease.

Embodiment 94 is the kit of any one of embodiments 83-93, further comprising a nuclease.

Embodiment 95 is the kit of any one of embodiments 83-94, further comprising a reverse transcriptase.

Embodiment 96 is the method of any one of embodiments 44-79, comprising (a) contacting the fixed biological sample with a substrate comprising a plurality of capture probes, wherein a capture probe of the plurality of capture probes comprises a capture domain that is capable of binding specifically to a ribosomal RNA (rRNA) from the FFPE biological sample; (b) staining and imaging the fixed biological sample; (c) de-crosslinking one or more crosslinks in the fixed biological sample; (d) extending an end of the capture probe using the rRNA specifically bound to the capture domain as a template, thereby generating an extended capture probe; (e) contacting a first detectable probe to the extended capture probe, wherein the first detectable probe comprises (i) a sequence that corresponds to a first sequence present in a 3′ region of the rRNA, and (ii) a first detectable label; (f) detecting a location of the first detectable probe on the substrate; (g) contacting a second detectable probe to the extended capture probe, wherein the second detectable probe comprises (i) a sequence that corresponds to a second sequence in the rRNA that is positioned 5′ relative to the first sequence in the rRNA, and (ii) a second detectable label; (h) detecting a location of the second detectable label on the substrate; and (i) comparing a stained image of the fixed biological sample and the location of the first detectable label and the second label on the substrate, thereby generating the spatial fragment DV heat map of the fixed biological sample.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition section headings, the materials, methods, and examples are illustrative only and not intended to be limiting.

What is claimed is:

1. A method of determining the presence of RNA of sufficient integrity suitable for downstream applications of a fixed biological sample, the method comprising:
(a) determining a spatial fragment distribution value (DV) number for the fixed biological sample, wherein the determining comprises:
(i) contacting the fixed biological sample with a substrate comprising a plurality of capture probes, wherein a capture probe of the plurality of capture probes comprises a capture domain that is capable of binding specifically to a ribosomal RNA (rRNA) from the fixed biological sample;
(ii) de-crosslinking one or more crosslinks in the fixed biological sample;
(iii) permeabilizing the fixed biological sample under conditions sufficient to hybridize the rRNA to the capture domain;
(iv) extending a 3′ end of the capture probe using the rRNA as a template, thereby generating an extended capture probe;
(v) hybridizing a first detectable probe to the extended capture probe, wherein the first detectable probe comprises (i) a sequence that corresponds to a first sequence present in a 3′ region of the rRNA, and (ii) a first detectable label; and
(vi) detecting the first detectable label, thereby determining a spatial fragment DV number of the first detectable label; and
(b) using the determined spatial fragment distribution value (DV) number to determine the presence of RNA of sufficient integrity suitable for downstream applications of the fixed biological sample.

2. The method of claim 1, further comprising determining the RNA integrity number score of the fixed biological sample and using the RNA integrity number score in combination with the determined DV number to determine the presence of RNA of sufficient integrity suitable for downstream applications of the fixed biological sample.

3. The method of claim 1, wherein extending the 3′ end of the capture probe comprises generating a single-stranded cDNA.

4. The method of claim 1, wherein the method further comprises, before step (b), a step of staining and imaging the fixed biological sample.

5. The method of claim 4, wherein the fixed biological sample is stained with hematoxylin and eosin.

6. The method of claim 1, wherein the rRNA is an 18S rRNA or 28S rRNA.

7. The method of claim 1, wherein the de-crosslinking step (a)(ii) comprises one or more of heating the fixed biological sample, the performance of a chemical reaction, the use of an enzyme, or the use of TE buffer, wherein the TE buffer has a temperature of about 65° C. to about 75° C., and is contacted with the fixed biological sample for about 30 minutes to about 90 minutes.

8. The method of claim 1, wherein the step of extending the end of the capture probe is performed in the presence of actinomycin D.

9. The method of claim 1, wherein the method further comprises treating the fixed biological sample with an RNase after step (a)(iv), and optionally, wherein the RNase is RNase H.

10. The method of claim 1, wherein the step of permeabilizing the fixed biological sample includes the use of a protease, and optionally, wherein the protease is pepsin or proteinase K.

11. The method of claim 1, wherein the fixed biological sample is removed after the extending in step (a)(iv).

12. The method of claim 1, further comprising:
hybridizing a second detectable probe to the extended capture probe, wherein the second detectable probe comprises: (i) a sequence that corresponds to a second sequence in the rRNA that is positioned 5′ relative to the first sequence in the rRNA, and (ii) a second detectable label; and
detecting the second detectable label, thereby determining a spatial fragment DV number of the second detectable label.

13. The method of claim 12, wherein the method further comprises hybridizing a third detectable probe to the extended capture probe, wherein the third detectable probe comprises (i) a sequence that corresponds to a third sequence in the rRNA that is positioned 5′ relative to the second sequence in the rRNA, and (ii) a third detectable label; and
detecting the third detectable label, thereby determining a spatial fragment DV number of the third detectable label.

14. The method of claim 13, wherein the first detectable label, the second detectable label, and the third detectable label is a fluorophore.

15. The method of claim 13, wherein the first detectable label, the second detectable label, and the third detectable label are different.

16. The method of claim 13, wherein the first detectable label, the second detectable label, and the third detectable label are the same.

17. The method of claim 12, wherein the method further comprises, a step of disassociating and removing the first detectable probe from the extended capture probe prior to hybridizing the extended capture probe with the second detectable probe.

18. The method of claim 13 wherein the method further comprises, a step of disassociating and removing the second detectable probe from the extended capture probe prior to hybridizing the extended capture probe with the third detectable probe.

19. The method of claim 1, wherein the first detectable probe detects a short extended capture probe, wherein the short extended capture probe comprises an extended capture probe of about 60 nucleotides or less from the 3′ end of the captured analyte.

20. The method of claim 12, wherein the second detectable probe detects a mid-length extended capture probe, wherein the mid-length extended capture probe comprises an extended capture probe from at least about 120 nucleotides to about 180 nucleotides from the 3′ end of the captured analyte.

21. The method of claim 13, wherein the third detectable probe detects a long extended capture probe, wherein the long extended capture probe comprises an extended capture probe from at least about 180 nucleotides to about 220 nucleotides from the 3′ end of the captured analyte.

22. The method of claim 1, wherein the spatial fragment DV number of the fixed biological sample comprises a number between 1 and 100.

23. The method of claim 22, wherein the spatial fragment DV number of the long extended capture probe comprises 60 or greater is indicative of RNA of sufficient integrity suitable for downstream applications.

24. The method of claim 2, wherein generating the RIN score for the fixed biological sample comprises a score between 1 and 10 and wherein the RIN score of 7 or greater is indicative of RNA of sufficient integrity suitable for downstream applications.

25. The method of claim 2, wherein the fixed biological sample comprises the spatial fragment DV number of the long extended capture probe of 60 or greater and the RIN score of less than 7.

26. The method of claim 1, wherein a downstream application comprises spatial transcriptomics.

27. The method of claim 1, wherein the fixed biological sample is a formalin-fixed paraffin-embedded biological sample, a PFA fixed biological sample, an acetone fixed biological sample, a tumor sample, an FFPE tissue section, a PFA tissue section, or an acetone fixed tissue section.

28. The method of claim 4, wherein the imaging and/or staining identifies a region of interest in the fixed biological sample.

29. The method of claim 25, wherein the fixed biological sample comprises the spatial fragment DV number of the long extended probe of 60 or greater and the RIN score of less than 7 is indicative of RNA of sufficient integrity for downstream applications.

30. The method of claim 1, wherein the spatial fragment DV number is an indication of RNA degradation in the fixed biological sample.
